(19) United States
(12) Patent Application Publication
(10) Pub. No.: US 2012/0271141 A1
(43) Pub. Date: Oct. 25, 2012

(54) SKIN SURFACE ELECTRODES

(75) Inventors: Richard J. Davies, Saddle River, NJ (US); Mary K. Davies, Saddle River, NJ (US)

(73) Assignee: Epi-Sci. LLC, Saddle River, NJ (US)

(21) Appl. No.: 13/502,439

(22) PCT Filed: Oct. 18, 2010
(86) PCT No.: PCT/US2010/053015

§ 371 Date: Jun. 7, 2012

Related U.S. Application Data
(60) Provisional application No. 61/279,471, filed on Oct. 21, 2009.

(51) Int. Cl. A61B 5/053

(52) U.S. Cl. 600/382; 600/387

(57) ABSTRACT

An apparatus (10) adapted to contact epithelial tissue, the apparatus comprising a cup (12) having a concave shape and adapted to be positioned on epithelial tissue, the cup capable of maintaining a reduced air pressure and holding a volume of flowable material; and an electrical support structure (14) comprising a support structure and a plurality of sensors, electrodes, or both (15) configured to interact with epithelial and subepithelial tissue.

### CROSS-REFERENCE TO RELATED APPLICATIONS

The present application claims the benefit of Application Ser. No. 61/279,471, filed Oct. 21, 2009, entitled SKIN SURFACE ELECTORDES, the disclosure of which is hereby incorporated herein by reference.

### BACKGROUND OF THE INVENTION

The present invention relates generally to the measurement or detection of electrical signals such as in abnormal or cancerous tissue, and more particularly, to the detection of changes in the electrophysiological characteristics of abnormal or cancerous tissue and to changes in those electrophysiological characteristics related to the functional, structural and topographic (the interaction of shape, position and function) relationships of the tissue during the development of malignancy. Measurements can be made in the absence or presence of pharmacological, hormonal or other chemical agents to reveal and accentuate the electrophysiological characteristics of abnormal or cancerous tissue.

Difficulty in detecting abnormal pre-cancerous or cancerous tissue before treatment options become non-viable is one of the reasons for the high mortality rate from cancer. Detecting the presence of abnormal or cancerous tissues is difficult, particularly where affected tissues are located beneath the skin surface, for example, deep within the body, thus requiring expensive, complex, invasive, and/or uncomfortable procedures. Thus, the use of detection procedures is often restricted or delayed until a patient experiences symptoms related to abnormal tissue. Many forms of cancers or tumors, however, require extended periods of time to attain a detectable size and thus to produce significant symptoms or indicate their presence in the patient. It is often too late for effective treatment by the time detection is performed with currently available diagnostic modalities.

Breast cancer is the most common malignancy affecting women in the Western World. The reduction in mortality for this widespread disease depends in significant part on early detection. The mainstay of early detection is X-ray mammography and clinical breast examination. Both are fraught with difficulties, including inaccuracy. For example, mammography has a lower sensitivity in women with dense breasts, and it is also unable to satisfactorily discriminate between morphologically similar benign and malignant breast tissue.

Clinical breast examinations are limited because lesions less than one centimeter are usually undetectable and larger lesions may be obscured by diffuse nodularity, fibrocystic change, or may be too deep in the breast to enable such clinical detection. Patients with positive mammographic or equivocal clinical findings often require biopsy to make a definitive diagnosis.

Accordingly, in view of the relatively poor specificity in diagnosing breast cancer, mammography and clinical breast examination can result in many positive mammographic findings or lesions detected on clinical breast examination which ultimately prove to be false positives, resulting in physical and emotional trauma for patients. Improved methods and technologies to accurately detect lesions and/or to identify patients who may need to undergo an invasive biopsy would reduce healthcare costs and avoid unnecessary diagnostic biopsies.

### BRIEF SUMMARY OF THE INVENTION

In a first embodiment, an apparatus for detecting electrophysiological characteristics in tissue may include a cup having a concave shape and adapted to be positioned on epithelial tissue, the cup capable of maintaining a reduced air pressure and holding a volume of flowable material; and an electrical support structure comprising a support structure and a plurality of sensors, electrodes, or both configured to interact with epithelial and subepithelial tissue.

In another embodiment, an apparatus for determining electrophysiological characteristics of subepithelial tissue, the apparatus comprising a cup adapted to contact epithelial tissue comprising a surface having a concave shape, having a volume enclosed by the cup and the epithelial tissue, the cup capable of maintaining a reduced air pressure and holding a volume of flowable material; and an electrical support structure comprising a connection between an instrument for measuring or recording electrical signals and a plurality of sensors, electrodes or both configured to interact with the epithelial and subepithelial tissue. The apparatus may further include an at least one port, and a reservoir comprising a volume of flowable material, in connection with a first port through which at least a portion of the flowable material may be transferred to the volume of the cup; an overflow reservoir; and a valve positioned on a path between the cup and the overflow reservoir for facilitating, maintaining or both, a reduced air pressure within the volume of the cup.

In yet another embodiment, an apparatus for detecting electrophysiological characteristics in breast tissue, the apparatus comprising a cup having a concave shape and adapted to be positioned over an area immediately surrounding a nipple of the breast, the cup capable of maintaining a reduced air pressure and holding a volume of flowable material within a volume defined by the concave shape of the cup and an area immediately surrounding the nipple; a reservoir, integral with the cup, comprising a volume of flowable material, the reservoir adapted to connect with the volume of the cup through a port through which at least a portion of the volume of flowable material may be transferred to the volume of the cup; an instrument for measuring or recording electrical signals; and a plurality of electrodes, sensors or both attachably configured in, on or adjacent to the cup, adapted to interact with the nipple and ductal and epithelial tissue of the breast.

In a further embodiment, an apparatus for detecting electrophysiological characteristics in subepithelial tissue, the apparatus comprising a cup adapted to contact epithelial tissue comprising a surface having a concave shape, having a volume enclosed by the cup and the epithelial tissue capable of both maintaining a reduced air pressure and holding a flowable material, and at least one port; a plurality of sensors attachably configured on and adjacent to the cup to contact the epithelial tissue and interact with the ductal epithelial tissue, subepithelial tissue and other deep tissue.

In an alternate embodiment, the apparatus may further include at least a second port associated with the cup. Alternatively, the apparatus may include a puncture seal comprising a portion of the surface of the cup. Alternatively, the apparatus may include a pump in connection with the reservoir to transfer at least a portion of the flowable material to the volume of the cup. Moreover, the cup may be deformable upon the generation of the reduced air pressure or upon application of pressure to an outer surface of the concave shape.

In yet a further embodiment, the present invention may include a method of detecting electrophysiological characteristics in subepithelial tissue, the method comprising contacting epithelial tissue with an apparatus comprising a cup having a concave shape and adapted to be positioned on epithelial tissue, the cup capable of maintaining a reduced air pressure and holding a volume of flowable material, and an electrical support structure comprising a support structure and a plurality of sensors, electrodes, or both configured to interact with epithelial and subepithelial tissue; and connecting the apparatus to an instrument for measuring or recording electrical signals from the apparatus.

In certain embodiments, the flowable material and/or the electrical support structure may be altered or changed to detect electrophysiological changes in the subepithelial tissue.

### BRIEF DESCRIPTION OF THE DRAWINGS

FIG. 1 illustrates a first embodiment of the apparatus.

FIG. 2 illustrates a cross-sectional view of the first embodiment of FIG. 1.

FIG. 3 illustrates an exploded view of a second embodiment of the apparatus.

FIGS. 4a and 4b illustrate various embodiments of electrical support structures of the apparatus.

FIG. 5 illustrates a further embodiment of an electrical support structure of the apparatus.

FIG. 6 illustrates yet another embodiment of an electrical support structure of the apparatus.

FIG. 7 illustrates another embodiment of an electrical support structure of the apparatus.

### DETAILED DESCRIPTION

Reference will now be made in detail to an embodiment of the invention, examples of which are illustrated in the accompanying drawings.

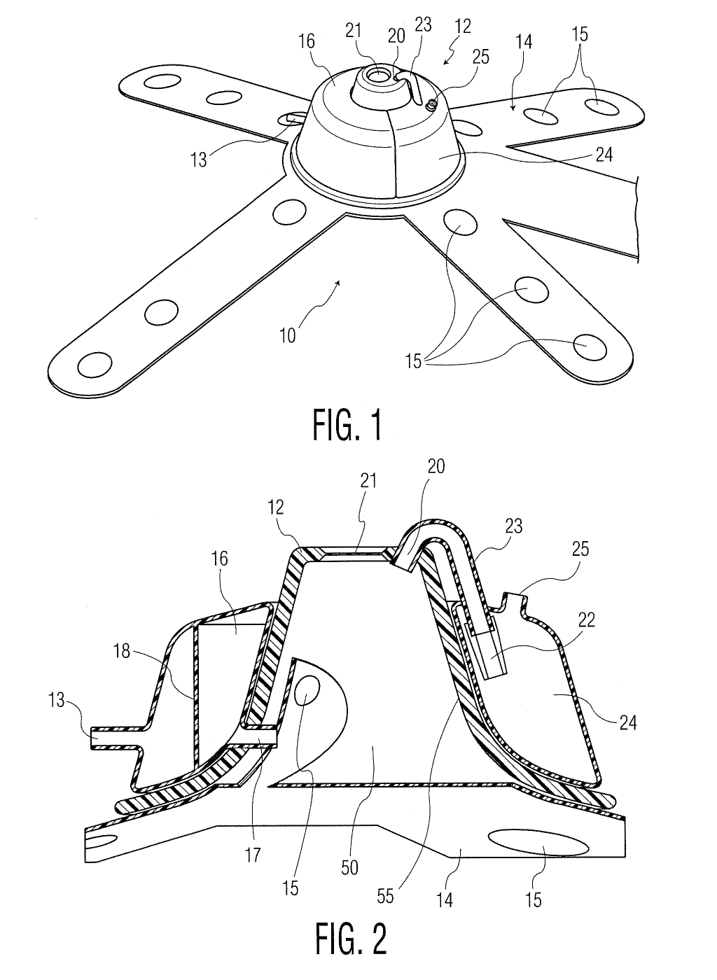

The present invention overcomes deficiencies associated with prior devices. In a first embodiment, as exemplified in FIG. 1, the apparatus 10 of the present invention includes generally a cup 12 and an electrical support structure 14. Apparatus 10 may be positioned on an epithelial tissue, such as the skin and/or nipple of a breast, and may be used to send and/or receive electrical signals through the epithelial tissue to measure, for example, density, of subepithelial tissue which may be tissue under the surface tissue (e.g., skin, nipple surface, etc.) and may include breast tissue, epithelial ductal tissue, deep tissue, and the like.

As illustrated in FIG. 2, the cup 12 may have an open volume 50 which is bounded by the inner surface 55 of cup 12. The cup 12 may have any required shape, such as conical, half spherical, or the like, and may further have a domed tip (which may assist in collecting any remaining air within volume 50 during the process of reducing air pressure, discussed below). The cup 12 may include at least one port, and may further include at least two ports, for example, a fill port 17 and an exhaust port 20. The cup 12 may include additional ports which may perform various functions. For example, cup 12 may include a manual drain port (not shown) positioned at the bottom of cup 12, when cup 12 is positioned on tissue, for manual draining of cup 12. Manual draining may be needed in the event of a problem with the electronics, pump, or other element of the cup, in which case the cup may have to be drained manually.

The fill port 17, as exemplified in FIG. 2, may connect the volume 50 of cup 12 with reservoir 16, which may contain a flowable material therein, such as for example physiological saline, which may optionally be prefilled into reservoir 16. The flowable material in reservoir 16 may be transferred to volume 50 through fill port 17. In one example, the reservoir 16 may be acted upon by a force generated by a reservoir deformable diaphragm 18, which is in turn acted upon by a pump (not shown) through pump port 13. Alternatively, the pump may directly interact with the reservoir to push or pull the flowable material from the reservoir 16 to the volume 50. Any pump suitable for acting on a deformable diaphragm may be used. Of course, any other technique for transferring a fluid from one receptacle to a different receptacle may be used to move the flowable material into volume 50 from reservoir 16 such as a manually operated air compressor or the like. Cup 12 may also include an exhaust port 20 coupled to passageway 23 which connects the volume 50 with exhaust/overflow reservoir 24 through passageway 23 and exhaust port 20. Passageway 23 may be a channel, piping, tubing, or any other structure which may allow the passage of a flowing material. A valve 22, such as a one-way valve known in the art, may be connected to passageway 23 to limit flow through passageway 23 in only one direction, for example, only in the direction of the exhaust/overflow reservoir 24. A single port into volume 50 may also be used in cup 12, and likewise, more than two ports may be used, depending upon the configuration of the passageway from the volume 50 to the other elements, for example, reservoirs 16 and 24.

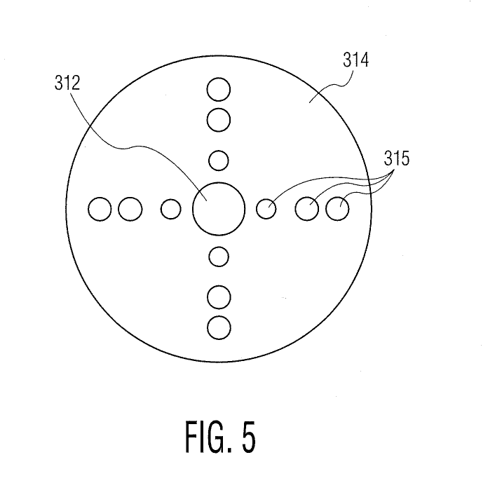

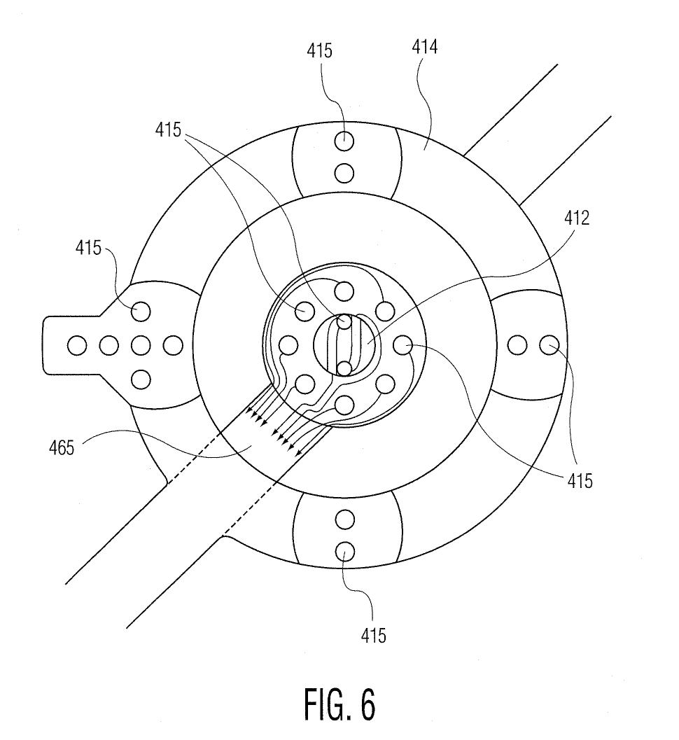

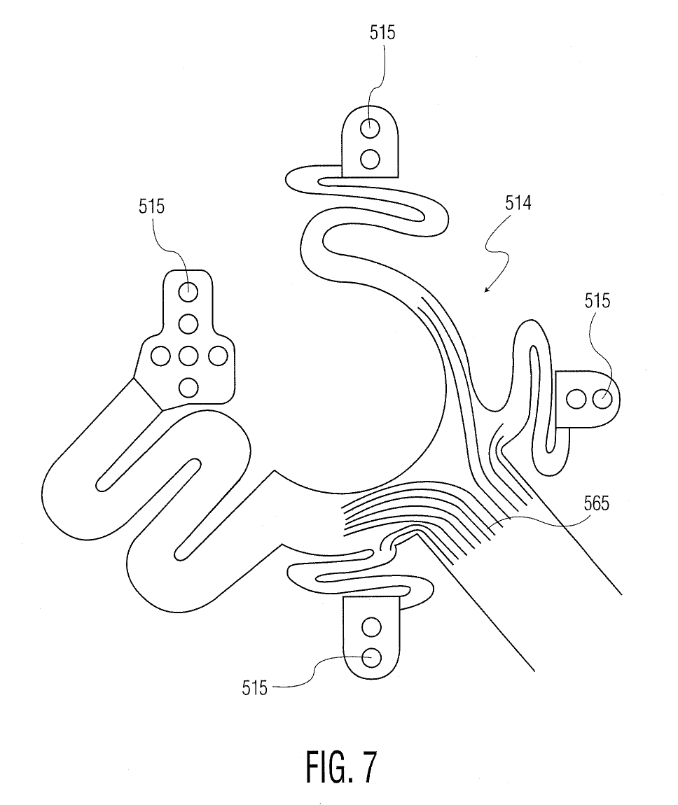

The electrical support structure 14 may, in one embodiment, as exemplified in FIGS. 1 and 2, be connected to cup 12 such that apparatus 10 is a single or integrated component including cup 12 and electrical support structure 14. The electrical support structure 14 may include a plurality of sensors 15 positioned at various places on the electrical support structure 14 to form an electrode array or sensor array. The electrical support structure 14, 214, shown in the figures, in particular FIG. 4, includes a plurality of “arms” extending from a central hub portion, which may be located on or in combination with cup 12. Of course, any other suitable shape for the electrical support structure 14, 214 is envisioned, for example single, double, triple, quadruple arms, or 6-, 7-, 8-armed, or the like. Alternatively, the plurality of sensors 315 can be positioned on a circular or ellipsoid shaped electrical support structure 314, such that it is a pad or disk, which may then be in combination with cup 312, an example of which is illustrated in FIG. 5. FIGS. 6 and 7 illustrate yet further alternative embodiments of an electrical support structure 414, 514. Support structure 414 of FIG. 6 is shaped as an annulus with additional structure located on or in combination with the cup 412 itself. FIG. 7 illustrates a support structure 514 having multiple extendable arms which allow a user of the apparatus to place the various sensors 515, located on the end portions of the various arms, at positions a distance away from the cup (not shown here), which may itself be positioned within an area enclosed or partially enclosed by at least two arms, such as for example, by the two larger arms as illustrated, or elsewhere on or adjacent to the support structure 514. In an alternate arrangement, the sensors 515 may further be positioned on the extendable portions of the arms and as such may be located on a portion of epithelial tissue intermediate to the end portions of the arms and the cup.

The plurality of sensors 15, 215, illustrated for example on FIGS. 1, 2 and 4a and b, may be positioned along the arms and/or within the volume 50 of cup 12, and may even be included on or adjacent to an adhesive film or foam 260 which may be positioned within cup 12. Alternatively, the adhesive film or foam 260 may not be positioned on the support structure 214, and may instead be positioned within cup 12, and thus the inner sensors 215 may be only attached to the wire connections 265 (which may be shaped as a straight length or as a “curl,” as illustrated) and be free of any adhesive material or other surface (aside, for example, from a paper backing, or the like, on which the support structure 214 is placed prior to use. The support structure 214 may be peeled from the paper backing and positioned on the tissue to be tested). In a further alternative embodiment, the inner sensors 215 may be directly applied to the cup 12, through for example a silk screen process or the like. In this variation, the cup and inner sensors 215 (e.g., within the circular central area of the four-armed support structure in FIG. 4a) may then be connected, through any known means such as an adhesive, to the rest of the support structure 214. In another embodiment illustrated in FIG. 6, the sensors 415 in and around cup 412 may be connected to wire connections 465, illustrated in part by the lines ending with arrows. The sensors 415 located around the annular portion of support structure 414 may also be connected to wire connections 465 (not shown).

The sensors 15, 215 should be placed on the arms, or the like, at various distances from the cup 12 depending on the area to be tested. In one example, when testing the breast, the sensors 15 may be located up to about 8 to about 10 cm from the cup 12 in order to test a significant portion or substantially the entire breast, although at least one sensor 15, 215 should be placed within the cup 12 to send and/or receive electrical signals directly through the nipple, affiliated ductal tissue of the nipple, and/or other tissue positioned within the cup 12. Of course, if the support structure 514 of FIG. 7 were used, the various electrodes 515 could be positioned further than about 8 to about 10 cm from the cup.

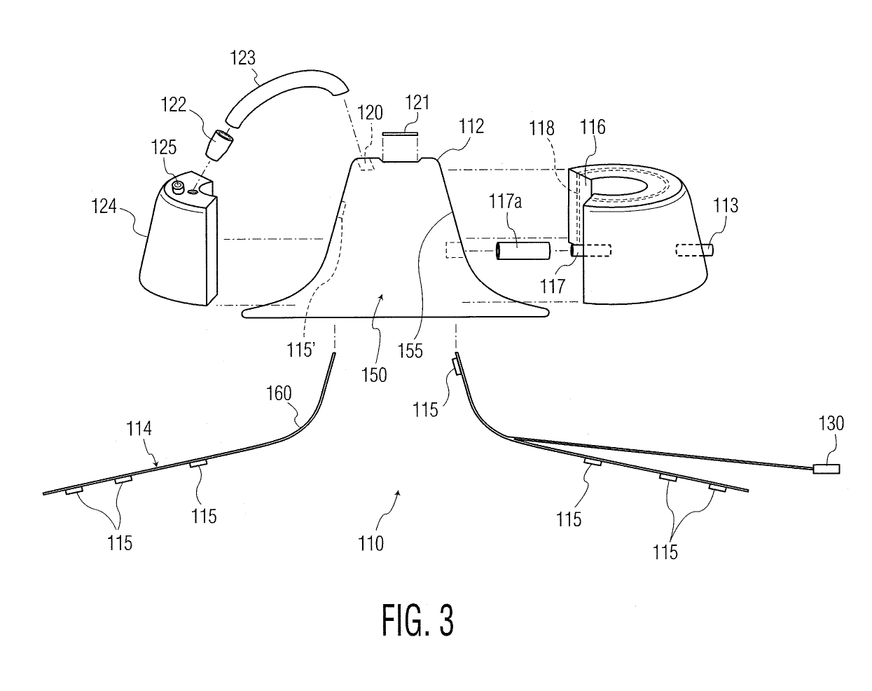

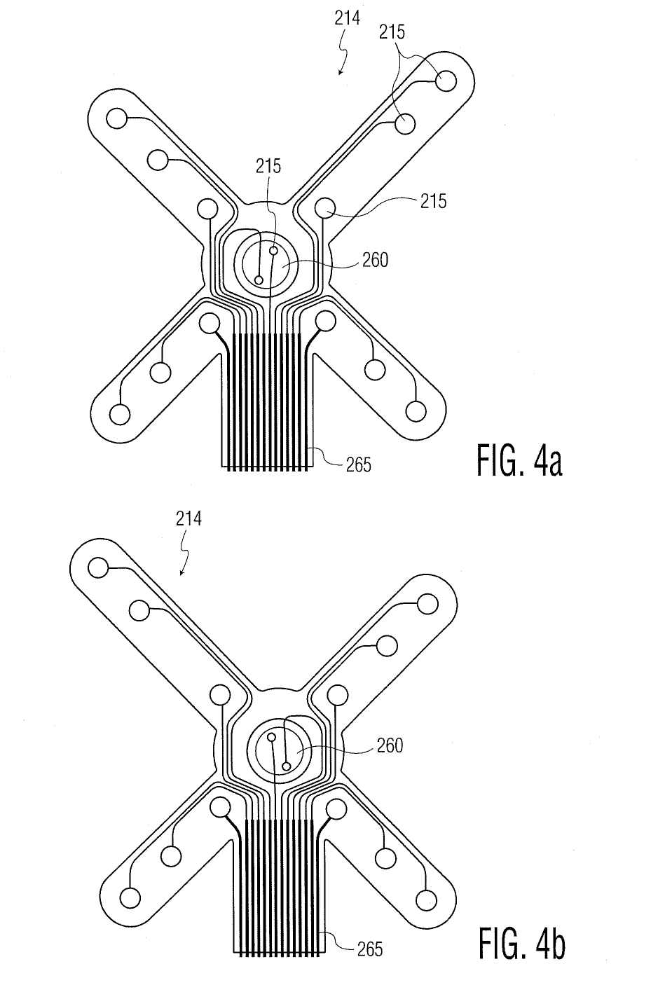

The sensor 15 positioned within the cup 12 may not physically contact the nipple, however, and may be positioned in the cup 12 such that the sensor 15 only contacts the flowable material and not the nipple tissue itself. The electrical support structure 14, 214 may further include an adhesive film or foam 60, or the like to promote adherence of support structure 14, 214 to the skin. Other alternative arrangements may be used, such as the portion of the support structure 14, 214 within or near cup 12 may have the adhesive film or foam 60, while the out-lying portions of support structure 14 may use tape or a different adhesive. Similarly, support structure 314, 414, 514 may utilize such adhesive films, foams or other substances over at least a portion of the circular, ellipsoid, or annular surface, or the surface of the ends of the elongated arms, including any area adjacent the cup 312, 412 to promote adhering contact between cup 312, 412 and support structure 314, 414. Each sensor 215 may be connected by wire connections 265, as illustrated in FIGS. 4a and b. The electrical support structure, through wire connections 265, may connect to an instrument (not shown) through an instrument cable 130 (for example, FIG. 3), which is typically an instrument which controls the apparatus 10 and electrical signals passing therethrough—hereinafter referred to as a control instrument. The wire connections and control instrument may be any known in the art.

While FIGS. 1 and 2 have been described above as illustrating a single assembly comprising the apparatus 10, apparatus 10 may also comprise a two-part assembly, or multiple part assembly, which may be assembled prior to use. For example, the cup 12 may be one part, and the electrical support structure 14 and ancillary elements such as the reservoirs 16 and 24, passageway 23 and the like may be the second part. Alternatively, the cup 12 and electrical support structure 14 may be the first part, while the ancillary elements such as the reservoirs 16 and 24, passageway 23 and the like may form a donut-shaped second part. The ancillary elements may be connected to cup 12 through the attachment of the reservoir 16 and passageway 23 to fill port 17 and exhaust port 20, respectively, such that the donut-shaped second part surrounds cup 12 and seats on the base of cup 12. In a further example, the electrical support structure 14 may be a separate piece from both cup 12 and the ancillary elements described above. Any other combination of multiple-part apparatus 10, 110 may also be used.

FIG. 3 illustrates an exploded view of a second embodiment of apparatus 110 which may include cup 112, reservoir 116, a plurality of sensors 115 (as part of electrical support structure 114 or off the support structure 114, as with sensor 115′), exhaust/overflow reservoir 124 and at least two ports—fill port 117 and exhaust port 120. In this embodiment, the reservoirs are illustrated to be separate structures from the cup 112, and as such the reservoirs may be positioned anywhere relative to the surface 55 of cup 12. In FIG. 3, the reservoirs 116 and 124 may be connected by passageways 117a and 123, respectively. The exemplary embodiment of FIG. 3 may also include such elements as puncture seal 121, one-way valve 122, air exit port 125, reservoir liner diaphragm 118, pump port 113 (and pump, not shown), and the like. The surface 155 of cup 112, bounding volume 150, may further include a covering of at least a portion of surface 155 of adhesive foam 160. Adhesive foam 160 may be any material which provides further frictional or adhesive attachment to a portion of tissue (not shown).

The plurality of sensors 115, as illustrated in FIG. 3, may be positioned anywhere along the electrical support structure 114 which may be suitable for interaction with the tissue (not shown). Moreover, at least one sensor 115 may be positioned on the adhesive film or foam 160, and may further be located within the volume 150 of cup 112 such that it is positioned either on the adhesive film or foam 160 within the volume 150 or on the surface 155 of cup 112 itself (also see FIG. 2, sensor 15 within volume 50 of cup 12). Alternatively, the sensor 115 may have the adhesive film or foam 160 located on it, which then provides for attachment to the surface 155 of cup 112. Furthermore, the inner surface 55 of cup 12 may include an adhesive layer on at least a portion of its surface for adherence to the sensor 15 (the sensor 15 may or may not include adhesive material on itself in this arrangement). This is further illustrated in FIG. 3 as shown by sensor 115′. As illustrated in FIGS. 3 and 4a and b, sensors 115, 215, may be connected to wire connections 265 which may, for example, connect sensors 115, 215 to an instrument cable 130 and a control instrument (not shown). Other embodiments are also envisioned, such as wireless connections between the sensors 115 and 215 and the control instrument, or other such electrical configurations known in the art. The sensors 15, 115, 215 themselves may be constructed as known in the art and may be manufactured using any suitable material including, for example, silver, silver chloride, UV dielectric, or any combination thereof. Sensors may be of any suitable size, for example, about 3 mm to about 9 mm.

The various embodiments illustrated in FIGS. 5-7 may also be constructed, positioned, and used in similar fashion as discussed above with reference to FIGS. 1-4a and b. Additionally, the various electrical support structures disclosed throughout may further be used with other nipple cup designs such as disclosed in U.S. Published App. No. 2004/0253652 (R. J. Davies), the disclosure of which is incorporated by reference herein as if fully set forth herein.

In operation, the apparatus 10, 110 (hereinafter, for simplicity, the embodiment of FIGS. 1 and 2 will be used as an example, though the following descriptions may also apply to other embodiments, such as the embodiments of FIGS. 3-7) may be positioned on a portion of tissue. In one embodiment, for example, the tissue may be a nipple on the breast of a human, whereby the apparatus 10 may be used to conduct a test for the presence of cancerous or pre-cancerous tissue within the breast. Other tissues may also be used with apparatus 10, though the shape of cup 12, in FIGS. 1 and 2, is specific to the use of apparatus 10 with a nipple. Thus, if other tissues are to interact with apparatus 10, a different shape of cup 12 may be necessary to better conform to the other tissue shape.

One example of an method of use of apparatus 10 may include the placement of the cup 12 over a nipple such that the nipple is positioned within volume 50. Cup 12 may form an airtight (or substantially airtight during the test period) connection with the nipple and the remaining volume 50 may be filled with a flowable material (not shown). Adhesive film or foam 160 (FIG. 3) or other adhesive or friction-causing material may be present on the undersurface of cup 12 to assist in holding the cup 12 in place over the nipple. Once the cup 12 is in place, the electrical support structure 14 may be secured to the area surrounding the nipple (of course, if the embodiment of FIG. 7 is used, the arms of support structure 514 may be extended to areas distant from the nipple) and connected to the control instrument, using an instrument cable 130 (FIG. 3) or the like, and the pump may be connected to the pump port 13. The flowable material may initially be located in reservoir 16, and once the cup 12 is positioned on the nipple, may transfer into the volume 50. One possible use may include the pump or like element, which may produce a force through pump port 13 onto diaphragm 18, which in turn may transfer the force to the flowable material which passes through fill port 17 and into the volume 50.

The flowable material may be any suitable material capable of conducting an electrical current, and may include liquids, gels or the like. As used in this description, a flowable material may be a medium that permits transmission of electrical signals between the surface being measured and the sensors 15, 215, particularly those positioned inside the volume 50 of cup 12. The material may further include any ionic concentration, pharmacological agent, hormone or other compound added to the material or otherwise introduced to the tissue under investigation, selected to provide further information about the condition of the tissue, if desired. In another embodiment the concentrations of agents may be changed using a flow through system. In addition, the impedance at varying subepithelial tissue depths and responses of, for example, DC potential and/or impedance to different concentrations of ions, drug, hormones, or other agent may be used to estimate the probability of cancerous tissue being present.

Flowable materials for use with the present inventions could include various electrolyte solutions such as physiologic saline (e.g. Ringers) with or without pharmacological agents. One preferable electrolyte solution to infuse into the ductal system will represent a physiological Ringer solution. Typically this consists of NaCl 6 g/L, KCl 0.075 g/L, CaCl₂ 0.1 g/L, NaHCO₃ 0.1 g/L, and smaller concentrations of sodium hyper and hypophosphate at a physiological pH of 7.4. Other electrolyte solutions may be used where the electrolyte comprises approximately 1% of the volume of the solution. Hypertonic or hypotonic solutions that are greater or less than 1% may be used in provocative testing of the epithelium and/or tumor. The concentration of Na, K and Cl will be adjusted under different conditions to evaluate the conductance and permeability of the epithelium. Different pharmacological agents such as amiloride (to block electrogenic sodium absorption), Forskolin (or similar drugs to raise cyclic-AMP) and hormones such as prolactin or estradiol can also be infused with the Ringer solution to examine the electrophysiological response of the epithelium and tumor to these agents. Similarly, the calcium concentration of the infusate will be varied to alter the tight junction permeability and measure the electrophysiological response of the epithelium to such manipulation. Dexamethasone may be infused to decrease the permeability of the tight junctions, and the electrophysiological response will be measured. In a further embodiment, the flowable material may be an electroconductive fluid or gel. In one example, the flowable material may be physiological saline, such as Normosol™ (Hospira Inc., Lake Forest, Ill.), which is a sterile, nonpyrogenic, isotonic solution of balanced electrolytes in water. Suitable electrolytes may include sodium chloride, sodium acetate, sodium gluconate, potassium chloride, magnesium chloride, or the like or any combination thereof. The flowable material may then provide a medium for application of a reduced air pressure within the cup and/or electrical contact to the nipple, which will be further explained below.

In another embodiment, the flowable material may be an electroconductive media which may include conductive fluids, creams or gels used with external or internal electrodes to reduce the impedance (resistance to alternating current) of the contact between the sensor 15 and the skin or epithelial surface. In the case of DC sensors it is also desirable that the flowable material results in the lowest DC offset at the sensor surface, or an offset that can be measured. The flowable material will often contain a hydrogel that will draw fluid and electrolytes from deeper layers of the skin to establish electrical contact with the sensor 15. Sensors that are used to pass current require flowable materials with high conductance. Usually this is accomplished by using flowable materials with high electrolyte content. The electrolytes frequently used are KCl (potassium chloride) because of the similar ionic mobility of these two ions in free solution, so that sensor polarization is less of a problem than when ions of different mobility are used. Other ions such as sodium may be used in flowable material formulations, and the higher electrolyte concentrations result in more rapid sensor equilibration. Such various types of flowable materials, particularly those including a chemical agent, are capable of effecting a physiological response in the subepithelial tissue, wherein the chemical agent may include a defined ionic concentration, a pharmacological agent, a hormone, or any combination thereof. Such various flowable materials may be used to provide various data wherein various flowable materials having different properties are used in series (one after another) to detect electrophysiological changes in the subepithelial tissue.

In situations where estimations will be made of the permeability of the epithelium to specific ions, the concentration of K (potassium) in the flowable material will be varied so that the conductance of the epithelium to potassium may be measured electrophysiologically. An enhancer or permeant may be added to the flowable material to increase the conductance of the underlying skin to the electrolyte in the flowable material. Other approaches to improving electrical contact and/or reducing surface skin impedance include mild surface abrasion with pumice and alcohol to reduce surface skin resistance, abrasive pads such as Kendall Excel electrode release liner (Tyco Health Care, Mansfield, Ma.), 3M Red Dot Trace Prep (3M Corporation, St. Paul, Minn.), cleaning the skin with alcohol, an automated skin abrasion preparation device that spins a disposable electrode to abrade the skin (QuickPrep system, Quinton, Inc., Bothell, Wash.), the use of ultrasound skin permeation technology (SonoPrep, Sontra Medical Corporation, Franklin, Mass.; U.S. Pat. No. 6,887,239, Elstrom et al.), or silicon microneedle array electrodes, which just reach, or may even penetrate, the stratum corneum to reduce skin surface resistance. (See, for example, P. Griss et al., IEEE Trans. on Biomedical Eng., 2002, 49 (6), 597-604) (For a comparison and discussion of several methods see also, Biomedical Instrumentation & Technology, 2006; 39: 72-77. The content of the patent and journal documents are incorporated herein by reference.) For example, microneedle electrodes, also known as spiked electrodes, may be used on any of the sensors 15, but will be most useful when used as a sensor 15 on at least one location on at least one arm of the support structure 14. In one configuration, each arm will include at least one microneedle electrode. The microneedle electrodes include nano-sized needles on the tissue-contacting surface of the electrode which are conductive and may penetrate the stratum corneum and enter into the viable epidermis. They are typically sized from about 15 um to about 200 um in length, and more specifically about 30 um to about 50 um, about 30 um in width, and may be made of metal, coated silicon or coated plastic. An electrode may typically have about 100 to about 10,000 microneedles on its tissue-contacting surface. Microneedle electrodes are described in the following U.S. Patent references: U.S. Pat. No. 6,622,035; 2007/0135729; 2004/0167422; U.S. Pat. No. 7,032,301 and PCT Application WO07/068433, all of which are incorporated by reference herein.

Although specific examples have been given of drugs and hormones that may be used in “challenge” testing of the epithelium, subepithelium and tumor, any agonist or antagonist of specific ionic transport, or tight-junctional integrity, known to be affected during carcinogenesis may be implemented, particularly when it is known to influence the electrophysiological properties of the epithelium, subepithelium or tumor.

As alluded to above, prior to the passage of the flowable material into the volume 50, the electrical support structure 14, including the plurality of sensors 15, may also be connected to the surrounding tissue (typically epithelial tissue or skin) around or in proximity to the nipple. The electrical support structure may be flexible, to conform to the surrounding tissue, and may have an underside which secures attachment to the tissue surrounding the nipple, which may be an adhesive, an adhesive film or foam 160 (FIG. 3), or any other suitable material which creates a frictional engagement between the electrical support structure 14 and the tissue to ensure the arms of the electrical support structure 14 remain attached to the tissue (but of course, can also be removed without creating excessive discomfort to the patient). Specific to FIG. 7, each arm may only have adhesive on the extreme ends of each arm, around and near the sensors 515, as it is not necessary to attach the entire elongated arm to the skin, though of course, the entire arm may include adhesive and may be adhered to the skin along its length if such circumstances require it. As is known in the art, a gel, or the like, may be placed under each of the plurality of sensors 15 on the arms to promote conductivity and provide comfort to the patient.

As the flowable material is pumped into the volume 50, air may be pushed out of the volume 50 through exhaust port 20, one-way valve 22 and out air exit port 25. A reduced air pressure, within volume 50, may be created, using the pump, for example, to draw the nipple further into the volume 50 while removing air from the volume 50. The “reduced air pressure,” formed within volume 50, may have an air pressure that is less than ambient air pressure. For example, the reduced air pressure may be a partial vacuum of any pressure below ambient pressure or a substantially complete vacuum. The reduced air pressure may be created, for example, through a short series of suction and release actions using the pump to draw a portion of the flowable material back into reservoir 16, although other methods may be used to create the reduced air pressure. For example, the reduced air pressure may have a pressure of about 100 mm Hg, which typically coincides with roughly 5 mL of flowable material pumped back out of volume 50 and into reservoir 16. Then, about 3 mL of flowable material may be pumped back into volume 50 to lower the pressure to about 20 mm Hg. This back-and-forth process may be repeated multiple times to remove air bubbles from volume 50, raise the nipple further into cup 12, and the like. Any air remaining in the volume 50 may be collected within the domed tip of the cup 12, and may be removed by use of a syringe, a duct, a valve, or the like.

The one-way valve 22 may assist in forming the reduced air pressure by allowing air to pass through exhaust port 20 and into exhaust reservoir 24, but preventing air to pass from the reservoir 24 and back into volume 50 through exhaust port 20. In this respect, one-way valve 22 may be a check valve or other structure of similar use. Moreover, exhaust reservoir 24 may be a smaller volume than reservoir 16, for example about 5 mL, and may be used to hold any flowable material which passes through exhaust port 20 during the filling and/or reduced air pressure processes, or at any other time. Any volume of air and/or flowable material which passes through exhaust port 20 in excess of the volume of exhaust reservoir 24 may pass through air exit port 25. However, air exit port 25 should be positioned on the exhaust reservoir 24 such that, under normal circumstances, excess flowable material remains within exhaust reservoir 24 and cannot leak out of air exit port 25, but the air removed from the volume 50 may still be released, if required.

The reduced air pressure provides numerous benefits including that the air is substantially or completely out of volume 50, that the flowable material takes up substantially or entirely the remaining volume 50 that is not taken up by the nipple, and that the cup 12 is secured to the tissue, among other reasons. In one example, the cup 12, once the reduced air pressure is applied, will have about 10 mL of volume which the flowable material can occupy.

With the cup 12 placed in this position, the plurality of sensors 15, attached to the electrical support structure 14 which is attached to the bottom of cup 12 as in

FIG. 1, may be positioned at various points on the surrounding tissue. At least one sensor 15 is also placed within the volume 50 of cup 12, on surface 55 or adhesive foam 160, such that this at least one sensor 15 is contacted by the flowable material which also contacts the nipple.

Once the reduced air pressure is obtained and stabilized, the control instrument may supply electrical signals through the plurality of sensors 15. The electrical signals pass through the sensors 15 and into the surface tissue, whether the nipple via the flowable material, the surrounding epithelial tissue, or both. The sensors 15 may then gather data of the deep tissue below the surface tissue (such as the subepithelial tissue, epithelial ductal tissue, or the like) which is obtained by passing the electrical signals through the surface tissue and into the deep tissue. The sensors 15 may pass the data back to the control instrument which may analyze the data or pass it to a further machine capable of processing and analyzing the data. The electrical signals may conduct tests and acquire data anywhere a sensor 15 is placed. For example, in FIGS. 1 and 4, the sensors 15, 215 are placed on the electrical support structure 14, 214 which has arms in an “X”-shaped pattern. The arms, in this pattern, allow electrical signals to be passed through all four quadrants of a breast (upper right, upper left, lower right, lower left), including directly though the nipple (which may be considered to be located approximately at the crossing of the “X”), to test substantially all areas of the breast. Alternatively, in this example, the breast may be split into greater divisions than quadrants (i.e., eight or ten “pie slices”) for increased data gathering. Thus, the electrical support structure 14 may have additional arms, or branched arms, or any configuration to divide the breast into additional pie slices. Of course, fewer arms may also be used for more generalized and broad testing. The electrical signals can be passed multiple times into a single sensor 15, 215, to obtain multiple readings on the same pie slice, other shape, or directly through the nipple. If a specific area of division is to be tested, then the arms of electrical support structure 14 may be altered to coincide with the desired area for testing.

In one embodiment of the use of apparatus 10, 110, the sensors 15, 115, 215 may receive various electrical signals from the control instrument. The sensors 15, 115, 215 located on the arms of the electronic support structure 14, 214 are connected to a frequency response analyzer (FRA) which may be interfaced with the patient through a biological impedance isolator. A sine wave at one or more selected frequencies, for example, about 50 Hz or about 60 Hz may then be passed between the sensors 15, 115, 215 on the cup 12, 112 (connected to the nipple through the flowable material) and the outermost sensors 15, 115, 215 on the arms at each quadrant of the breast which may be located roughly 8-10 cm from the cup 12, 112. The impedance may then be measured in a sequential manner between the sensors 15, 115, 215 on cup 12, 112 and the outermost sensors 15, 115, 215 at each quadrant of the breast. This measurement may then be used to obtain the average tissue density of the four quadrants of the breast using the methods as disclosed in U.S. Ser. No. 12/316,032, incorporated herein by reference. This test may be replicated as many times as necessary and at other frequencies (e.g., about 10 Hz, about 100 Hz, about 1,000 Hz, about 10,000 Hz, using DC or the like). The sensors 15, 115, 215 on the arms may also be at various distances from the cup 12, 112 to obtain readings all along each quadrant (e.g., sensors may be at about 4 cm, 7 cm, 8 cm, 10 cm, etc.). In one method of using the apparatus, the operator may use various electrical connections having different properties, used in series (one after the other), to detect electrophysiological changes in the subepithelial tissue including altering the voltage, current, location of sensors, or any combination thereof. The density readings, for example, are then compared and the presence of pre-cancerous or cancerous tissue may be illustrated by a different reading of tissue characteristics, correlated with, for example, density in one quadrant, for example, than in the other quadrants or from a previous or expected value for a patient.

Once the test is complete whereby the electrical signals and data collection are completed, the operator may puncture the puncture seal 21 to release the reduced air pressure and empty the volume 50 of the flowable material. Puncture seal 21 may be punctured with a built-in mechanism or a hand-held object, such as a pin, knife, scalpel, or the like. The flowable material may then empty directly out of the puncture seal 21 or may pass back through to the reservoir 16. Alternatively, the pump may be used to increase pressure in the cup 12, to force the flowable material from the cup. Alternatively, the puncture seal 21 may be another structure, such as a rubber stop, valve, gate, or the like.

This use may be conducted on only a single breast/nipple or may be done on both breasts/nipples.

The apparatus 10, 110 may include other elements in addition to those discussed above. For example, various elements of apparatus 10, 110 may be made of different materials. Cup 12, 112 may be of a clear polymer or plastic to enable the operator to view inside the cup 12, 112 during the test. Either or both of the reservoirs may also be of a clear polymer plastic to enable viewing of the flowable material. Furthermore, the electrical support structures 14, 114, 214 may be made of polymer plastic which may be flexible to allow it to conform to the tissue, deform under pressure of the reduced air pressure and/or deform under application of pressure by the user to the outer surface of the concave shape of cup 12. Likewise, electrical support structure 14, 114, 214 can be similar to a tape having an adhesive underside. Other materials may also be used which are suitable for the purposes and uses discussed herein.

Apparatus 10, 110 may be disposable, or at least a portion may be disposable, or may be nondisposable and reusable. If apparatus 10, 110 is disposable, then puncture seal 21 may be constructed to be irreversibly breakable and flowable material may flow out of apparatus 10, 110. Apparatus 10, 110 may further be of a one-piece, two-piece or multiple piece design. Apparatus 10, 110 may then be removed from the tissue and disposed. If apparatus 10, 110 has only a disposable cup 12, then the seal may or may not be irreversibly breakable, and flowable material may either flow out of apparatus 10, 110 or back into reservoir 16 for later repeated use. In this embodiment, the cup 12 (FIGS. 1 and 2) may be one part (with or without electrical support structure 14) and the donut-shaped second part may be removable from cup 12 to be reused, and cup 12 may be disposed. In the further embodiment where the entire apparatus 10, 110 may be reusable, the puncture seal 21 may be reversibly breakable, meaning the seal may be opened to release the reduced air pressure, but may be re-sealed for the next test. In this embodiment, the puncture seal 21 may instead be, for example, a manually releasable check valve, plug or the like. The flowable material may, upon release of the puncture seal 21, flow back to reservoir 16, 116. Of course, any reusable portion of apparatus 10, 110 should comply with all sanitary requirements and should be capable of undergoing sterilization.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claim.

1. An apparatus for determining electrophysiological characteristics of subepithelial tissue, the apparatus comprising a cup adapted to contact epithelial tissue, the cup comprising a surface having a concave shape, having a volume enclosed by the cup and the epithelial tissue, the cup capable of maintaining a reduced air pressure and holding a volume of flowable material; and an electrical support structure comprising a connection between an instrument for measuring or recording electrical signals and a plurality of sensors, electrodes or both configured to interact with the epithelial and subepithelial tissue.

2. The apparatus of claim 1, wherein the cup further comprises at least one port.

3. The apparatus of claim 2, wherein the cup further comprises a reservoir, comprising a volume of the flowable material, in connection with a first port through which at least a portion of the flowable material may be transferred to the volume of the cup.

4. The apparatus of claim 3, further comprising a pump in connection with the reservoir to transfer the at least a portion of the flowable material to the volume of the cup.

5. The apparatus of claim 3, wherein the cup further comprises an overflow reservoir.

6. The apparatus of claim 5, wherein the cup further comprises a valve positioned on a path between the cup and the overflow reservoir for facilitating, maintaining or both, a reduced air pressure within the volume of the cup.

7. The apparatus of claim 3 further comprising at least a second port, wherein the second port is positioned between the cup and the valve.

8. The apparatus of claim 1, wherein one or more flowable materials having identified chemical, electrical or physiological properties are used to effect electrophysiological changes in the subepithelial tissue.

9. The apparatus of claim 1, wherein the flowable material is a chemical agent, capable of effecting a physiological response in the subepithelial tissue, the chemical agent including a defined ionic concentration, a pharmacological agent, a hormone, or any combination thereof.

10. The apparatus of claim 8, wherein at least two flowable materials are used in series.

11. The apparatus of claim 1, further comprising a puncture seal comprising a portion of the surface of the cup.

12. The apparatus of claim 1, wherein at least one electrical condition or property is impressed on the electrodes or sensors in order to effect electrophysiological changes in the subepithelial tissue including altering the voltage type or level, current type or level, location of sensors, or any combination thereof.

13. The apparatus of claim 1, wherein the cup is deformable upon the generation of the reduced air pressure or upon application of pressure to an outer surface of the concave shape.

14. The apparatus of claim 1, wherein at least one of the plurality of sensors, electrodes or both is positioned within the volume enclosed by the cup and the epithelial tissue.

15. The apparatus of claim 1, wherein the electrical support structure is configured to have a shape of a central hub with multiple radial arms extending therefrom, an annulus or a circular or ellipsoid disk.

16. The apparatus of claim 15, wherein the plurality of sensors, electrodes or both are positioned on the electrical support structure substantially throughout the shape of the electrical support structure.

17. The apparatus of claim 1, wherein at least one of the plurality of sensors, electrodes or both comprises a spiked electrode.

18. An apparatus for detecting electrophysiological characteristics in breast tissue, including nipple ducts and subepithelial tissue, the apparatus comprising a cup having a concave shape and adapted to be positioned over an area immediately surrounding a nipple of the breast, the cup capable of maintaining a reduced air pressure and holding a volume of flowable material within a volume defined by the concave shape of the cup and an area immediately surrounding the nipple; a reservoir, integral with the cup, comprising a volume of flowable material, the reservoir adapted to connect with the volume of the cup through a port through which at least a portion of the volume of flowable material may be transferred to the volume of the cup; an instrument for measuring or recording electrical signals; and a plurality of electrodes, sensors, or both attachably configured in, on, or adjacent to the cup, adapted to interact with the nipple, ductal and epithelial tissue of the breast.

19. The apparatus of claim 18, further comprising a pump in connection with the reservoir to transfer the at least a portion of the flowable material to the volume of the cup.

20. The apparatus of claim 19, wherein the cup further comprises an overflow reservoir.

21-32. (canceled)
